(19) United States
(12) Patent Application Publication
(10) Pub. No.: US 2024/0153622 A1
(43) Pub. Date: May 9, 2024

(54) MANAGEMENT APPARATUS, MANAGEMENT SYSTEM, TERMINAL DEVICE, AND NON-TRANSITORY COMPUTER-READABLE STORAGE MEDIUM STORING MANAGEMENT PROGRAM FOR MANAGING WORK ON LAVATORY EQUIPMENT

(71) Applicant: LIXIL Corporation, Tokyo (JP)

(72) Inventors: Shoji OGAWA, Tokyo (JP); Hiroki Shigetomo, Tokyo (JP); Toshiki Hashida, Tokyo (JP)

(73) Assignee: LIXIL Corporation, Tokyo (JP)

(21) Appl. No.: 18/502,669

(22) Filed: Nov. 6, 2023

(30) Foreign Application Priority Data
Nov. 9, 2022 (JP) 2022-179313

(51) Int. Cl. G16H 40/40; G06T 11/00

(52) U.S. Cl. CPC G16H 40/40; G06T 11/00

(57) ABSTRACT

In a management apparatus, an information acquirer acquires information from at least one of lavatory equipment and a sensor installed in a lavatory space. A necessity calculator calculates a work necessity indicating a degree of necessity of work on the lavatory equipment. A generator generates list information for displaying a list of sets of the lavatory equipment and the work necessity for the lavatory equipment. A transmitter transmits the list information to an external terminal device used by a person in charge of the work for the lavatory equipment.

### CROSS-REFERENCE TO RELATED APPLICATION

This application claims priority to Japanese Patent Application No. 2022-179313 filed on Nov. 9, 2022, the entire contents of which is incorporated by reference.

### FIELD

The present disclosure relates to a management apparatus, a management system, a terminal device, and a non-transitory computer-readable storage medium storing a management program for managing work on lavatory equipment.

### BACKGROUND

Lavatories are essential installations both in houses where people live and in facilities used by people. Since the lavatory is an installation which tends to generate dirt and odors, it is important to maintain and manage the cleanliness of the lavatory in order to make the house or facility a comfortable space. In particular, lavatories installed in commercial facilities, entertainment facilities, complex facilities, passenger station facilities, and the like used by large numbers of people need to be appropriately maintained and managed also from a viewpoint of public health.

A cleaning company entrusted with cleaning of lavatories by a manager of a facility patrols and cleans all lavatories several times a day according to a determined manual. In the case of a large-scale facility, the number of lavatories is several hundred, but it takes the time and effort of tens of persons in charge to clean all the lavatories.

Examples of the related art include Japanese Unexamined Patent Application Publication No. 2003-242230.

### SUMMARY

As described above, the work for maintaining and managing lavatories installed in the facility relies on a human-wave strategy by a large number of persons in charge of the work, and requires a large amount of labor and cost. Improvement of work efficiency for lavatory equipment is required.

Considering such an issue, an object of the present disclosure is to provide a technique for appropriately managing work on lavatory equipment.

In order to solve the above issues, according to some embodiments of the present disclosure, a management apparatus includes an information acquirer that acquires information from at least one of lavatory equipment and a sensor installed in a lavatory space, a necessity calculator that calculates a work necessity indicating a degree of necessity of work on the lavatory equipment based on the acquired information, a generator that generates list information for displaying a list of sets of the lavatory equipment and the work necessity for the lavatory equipment, and a transmitter that transmits the list information to an external terminal device used by a person in charge of the work for the lavatory equipment.

In some embodiments of the present disclosure, a management system includes a terminal device that is used by a person in charge of work on lavatory equipment installed in a lavatory space, and a management apparatus that manages the work on the lavatory equipment. The management apparatus includes an information acquirer that acquires information from at least one of the lavatory equipment and a sensor installed in the lavatory space, a necessity calculator that calculates a work necessity indicating a degree of necessity of work on the lavatory equipment based on the acquired information, a generator that generates list information for displaying a list of sets of the lavatory equipment and the work necessity for the lavatory equipment; and a transmitter that transmits the list information to the terminal device. The terminal device includes a presenter that presents, to the person in charge of the work, the list information transmitted from the management apparatus.

According to some embodiments of the present disclosure, a terminal device includes a presenter that presents, to a person in charge of work, list information transmitted from a management apparatus that generates the list information for displaying a list of sets of lavatory equipment installed in a lavatory space and a work necessity indicating a degree of necessity of the work on the lavatory equipment based on information acquired from at least one of the lavatory equipment and a sensor.

Any combinations of the above components and modifications of the expressions of the present disclosure in methods, apparatuses, systems, recording media, computer programs, and the like are also effective as embodiments of the present disclosure.

### BRIEF DESCRIPTION OF THE DRAWINGS

Embodiments will now be described, by way of example only, with reference to the accompanying drawings which are meant to be exemplary, not limiting, and wherein like elements are numbered alike in several FIGURES, in which:

FIG. 1 is a diagram illustrating a configuration of a management system, according to some embodiments;

FIG. 2 is a functional block diagram illustrating a configuration of a management apparatus, according to some embodiments;

FIG. 3 is a functional block diagram illustrating a configuration of a person-in-charge terminal, according to some embodiments;

FIG. 4 is a sequence diagram illustrating processing of the management system, according to some embodiments;

FIG. 5 is a diagram illustrating an example of a display screen displayed by the person-in-charge terminal, according to some embodiments;

FIG. 6 is a diagram illustrating an example of the display screen displayed by the person-in-charge terminal, according to some embodiments;

FIG. 7 is a diagram illustrating an example of a display screen displayed by the person-in-charge terminal, according to some embodiments;

FIG. 8 is a diagram illustrating an example of a display screen displayed by the person-in-charge terminal, according to some embodiments;

FIG. 9 is a diagram illustrating an example of a display screen displayed by the person-in-charge terminal, according to some embodiments;

FIG. 10 is a diagram illustrating an example of a display screen displayed by the person-in-charge terminal, according to some embodiments;

FIG. 11 is a diagram illustrating an example of a display screen displayed by the person-in-charge terminal, according to some embodiments;

FIG. 12 is a diagram illustrating an example of a display screen displayed by the person-in-charge terminal, according to some embodiments;

FIG. 13 is a diagram illustrating an example of a display screen displayed by the person-in-charge terminal, according to some embodiments; and

FIG. 14 is a diagram illustrating tenth example of a display screen displayed by the person-in-charge terminal.

### DETAILED DESCRIPTION

In some embodiments of the present disclosure, a technique for managing work on lavatory equipment will be described. In a management system of some embodiments, a person in charge of work on lavatory equipment can work on the lavatory equipment determined to have a high work necessity by a management apparatus, instead of regularly patrolling all pieces of the lavatory equipment. As a result, it is possible to eliminate waste of working on unused lavatory equipment many times every day and to appropriately work on the lavatory equipment, so that it is possible to greatly improve work efficiency for a plurality of pieces of lavatory equipment.

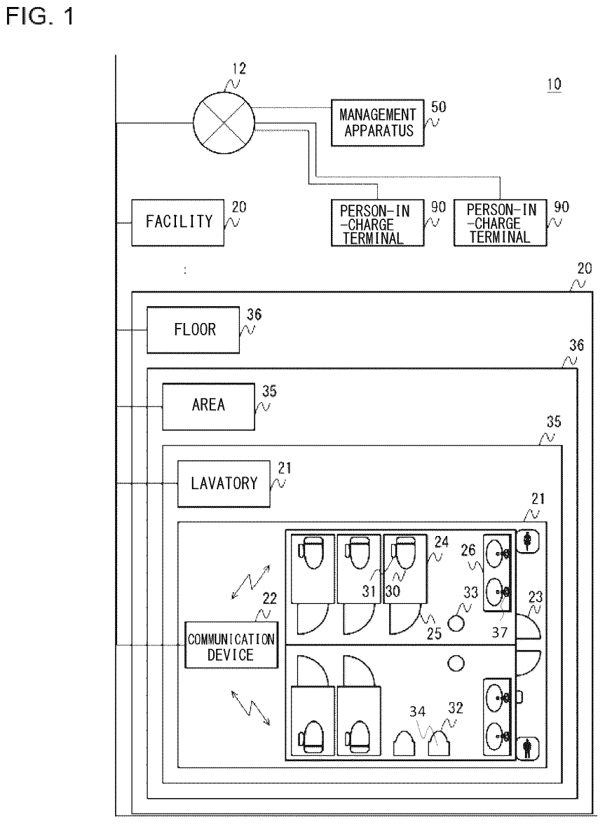

FIG. 1 illustrates a configuration of a management system according to some embodiments. A management system 10 includes a management apparatus 50 that manages work of a plurality of pieces of lavatory equipment 40 installed in a lavatory 21 of a facility 20, a communication device 22 that transmits information from at least one of the lavatory equipment 40 and a sensor 33 installed in the lavatory 21 to the management apparatus 50, a person-in-charge terminal 90 of a person in charge of work on the lavatory equipment 40, and the Internet 12 as an example of communication means for communicating between these devices. The lavatory 21 in some embodiments is an example of a lavatory space. The person-in-charge terminal 90 in some embodiments is an example of an external terminal device.

The facility 20 is hierarchically managed in a plurality of hierarchies. The facility 20 has one or more floors 36, and the floor 36 has one or more areas 35. One or more lavatories 21 are provided in each area 35. The lavatory 21 is provided with the sensor 33, a door 23 of the lavatory 21, one or more private rooms 24, a door 25 of each private room 24, and the lavatory equipment 40 having a water receiver (not illustrated) that receives water discharged from a water discharger. The lavatory equipment 40 in the present embodiment includes a washbasin 26, a toilet 30 installed in each private room 24, and a urinal 32 installed in a male lavatory 21. The washbasin 26 includes a water faucet and a water receiver that receives water discharged from the water faucet, and includes, as necessary, a liquid soap supply device that supplies a liquid soap, a disinfectant supply device that supplies a disinfectant, and the like (neither is illustrated). In the private room 24 provided with the toilet 30, toilet paper is provided, and a lavatory seat cleaner supply device, a trash box, and the like are provided as necessary. The toilet 30 may be a Japanese toilet or a Western toilet. The lavatory equipment 40 includes controllers 31, 34, and 37 for a user of the lavatory equipment 40 to input an operation instruction to the lavatory equipment 40. Each of the controllers 31, 34, and 37 is configured to be able to transmit operation information indicating that the lavatory equipment 40 has operated, to the management apparatus 50 via the communication device 22 in response to the operation instruction input by a user. The sensor 33 is configured to be able to transmit detection information to the management apparatus 50 via the communication device 22.

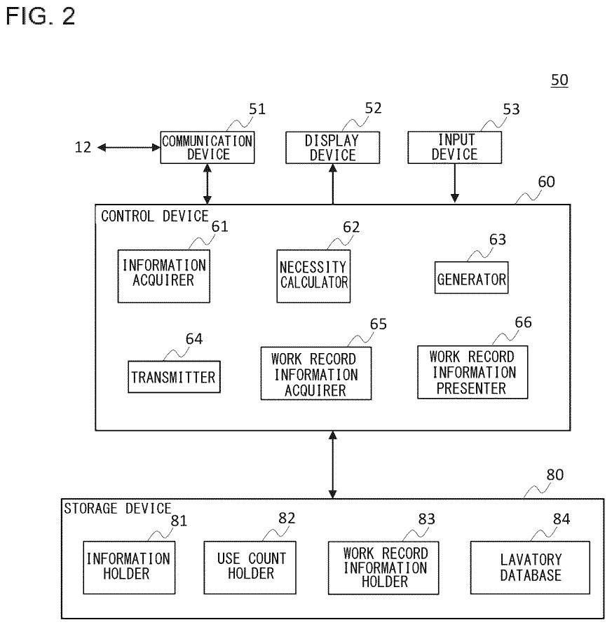

FIG. 2 is a functional block diagram illustrating a configuration of the management apparatus 50 according to some embodiments. The management apparatus 50 includes a communication device 51, a display device 52, an input device 53, a storage device 80, and a control device 60. The management apparatus 50 may be a server device, a device such as a personal computer, or a portable terminal such as a portable phone terminal, a smartphone, or a tablet terminal.

The communication device 51 controls communication with other devices. The communication device 51 may communicate by any wired or wireless communication scheme. The communication device 51 communicates with the communication device 22 via the Internet 12 and receives information from at least one of the lavatory equipment 40 and the sensor 33. The communication device 51 also communicates with the person-in-charge terminal 90 via the Internet 12.

The display device 52 displays a screen generated by the control device 60. The display device 52 may be a liquid crystal display device, an organic EL display device, or the like. The input device 53 transmits an instruction input by a user of the management apparatus 50 to the control device 60. The input device 53 may be a mouse, a keyboard, a touch pad, or the like. The display device 52 and the input device 53 may be mounted as a touch panel.

The storage device 80 stores programs, data, and the like used by the control device 60. The storage device 80 may be a semiconductor memory, a hard disk, or the like. The storage device 80 includes an information holder 81, a use count holder 82, a work record information holder 83, and a lavatory database 84.

The information holder 81 holds information acquired from at least one of the lavatory equipment 40 and the sensor 33. The use count holder 82 holds the number of times of using the lavatory equipment 40 as a work target. The work record information holder 83 holds information indicating a work record of the lavatory equipment 40 as the work target. The lavatory database 84 holds information regarding the lavatory equipment 40 as the work target. The lavatory database 84 holds, for example, information such as the number, positions, floors, and plan views of the lavatories 21, the number and positions of the private rooms installed in the lavatory 21, and the installation location, the installation floor, the number of installations, the type, the elapsed time from the installation, the use frequency, the work frequency, the manufacturing date, the manufacturing number, and the like of pieces of the lavatory equipment 40.

The control device 60 includes an information acquirer 61, a necessity calculator 62, a generator 63, a transmitter 64, a work record information acquirer 65, and a work record information presenter 66. These components are realized by a CPU, a memory, another LSI, or the like of any computer in terms of hardware, and are realized by a program loaded in the memory or the like in terms of software. Here, functional blocks realized by cooperation of the hardware and the software are illustrated. Therefore, it is understood by those skilled in the art that these functional blocks can be realized in various forms such as only hardware or a combination of hardware and software.

The information acquirer 61 acquires information from at least one of the lavatory equipment 40 and the sensor 33 via the communication device 22 and stores the information in the information holder 81.

The necessity calculator 62 calculates the number of times of use of the lavatory equipment 40 based on detection information and updates the number of times of use stored in the use count holder 82. The necessity calculator 62 calculates a work necessity indicating a degree of necessity of work on the lavatory equipment 40. The work here includes cleaning work, replenishment work of a consumable, and removal work of an abnormality. The replenishment of a consumable here includes, for example, replenishment of toilet paper, replenishment of a disinfectant, replenishment of a liquid soap to a liquid soap supply device (not illustrated) such as an automatic soap provided in the washbasin 26, and the like. The abnormality here includes, for example, clogging of the water receiver of the lavatory equipment 40, a failure or abnormality of a shower lavatory device (for example, a buttock cleaning water nozzle, a bidet cleaning water nozzle, a drying device, a deodorizing device, and a heated lavatory seat) provided in the toilet 30, a failure or abnormality of a flush device (not illustrated) of the toilet 30 and the urinal 32, a failure or abnormality of the water faucet or the liquid soap supply device of the washbasin 26, and the like. The work necessity and a calculation method thereof will be described later.

The generator 63 generates list information for displaying a list of sets of the lavatory equipment 40 and the work necessity of the lavatory equipment 40 based on the calculated work necessity. The list information itself may have an image displaying the list of the sets. Alternatively, an application for creating an image displaying the list of the sets based on the work necessity may be installed on the person-in-charge terminal 90 in advance, and the list information may cause the person-in-charge terminal 90 to create an image displaying a list of the sets based on the work necessity.

The transmitter 64 transmits the list information to the person-in-charge terminal 90.

The work record information acquirer 65 acquires work record information from the person-in-charge terminal 90 and stores the work record information in the work record information holder 83.

The work record information presenter 66 reads the work record information held in the work record information holder 83 and presents the work record information to a manager of the facility 20, a manager of the work, another person in charge of the work, and the like.

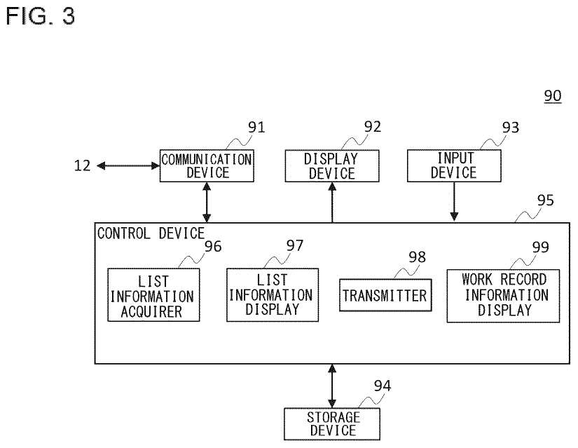

FIG. 3 is a functional block diagram illustrating a configuration of the person-in-charge terminal 90 according to the embodiment. The person-in-charge terminal 90 includes a communication device 91, a display device 92, an input device 93, a storage device 94, and a control device 95. The person-in-charge terminal 90 may be a device such as a personal computer, or may be a portable terminal such as a portable phone terminal, a smartphone, or a tablet terminal.

The communication device 91 controls communication with other devices. The communication device 91 may communicate by any wired or wireless communication scheme. The communication device 91 communicates with the management apparatus 50 via the Internet 12.

The display device 92 displays a screen generated by the control device 95. The display device 92 may be a liquid crystal display device, an organic EL display device, or the like. The input device 93 transmits an instruction input by the person in charge of work to the control device 95. The input device 93 may be a mouse, a keyboard, a touch pad, or the like. The display device 92 and the input device 93 may be mounted as a touch panel. The storage device 94 stores programs, data, and the like used by the control device 95.

The control device 95 includes a list information acquirer 96, a list information display 97, a transmitter 98, and a work record information display 99. These components can also be realized in various forms such as only hardware or a combination of hardware and software.

The list information acquirer 96 acquires the list information from the management apparatus 50. The list information display 97 displays the list information acquired by the list information acquirer 96 on the display device 92.

When the person in charge of the work has completed the work, the transmitter 98 transmits work record information including a message that the work has been completed, the content of the work, and the like to the management apparatus 50.

The work record information display 99 acquires work record information by another person in charge of work from the management apparatus 50 and displays the acquired work record information on the display device 92.

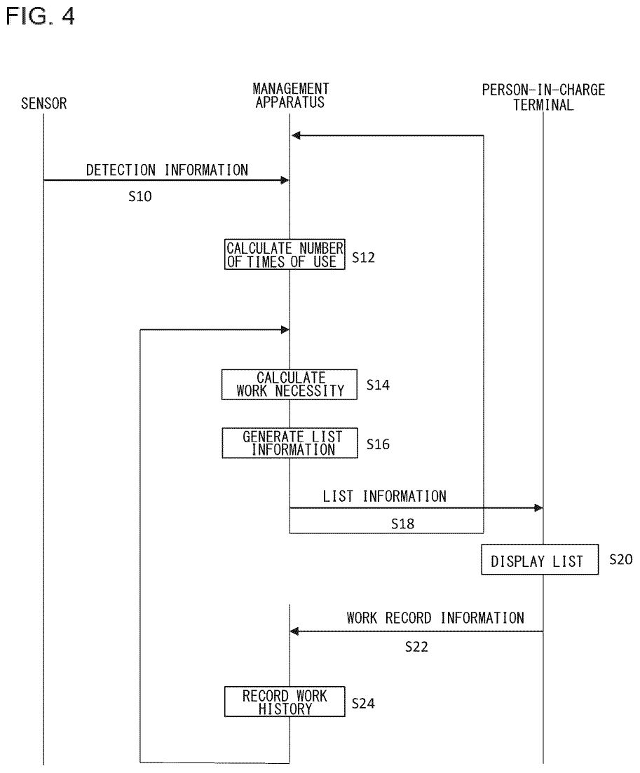

FIG. 4 is a sequence diagram illustrating processing of the management system 10 according to some embodiments.

In Step S10, the information acquirer 61 acquires detection information from the sensor 33 via the communication device 22. The sensor 33 may be, for example, an opening/closing sensor of the door 25 of the private room 24, a sensor that detects locking/unlocking of a key installed in the door 25, a pyroelectric sensor, a human sensor, a temperature sensor, a seating sensor of the toilet 30, a drain sensor, a clogging sensor, an odor sensor, a dirt sensor, a sound sensor, or the like. In the example of FIG. 4, the management apparatus 50 acquires the detection information from the sensor 33, but may acquire operation information from the lavatory equipment 40.

In Step S12, the necessity calculator 62 calculates the number of times of use of each piece of lavatory equipment 40 based on the acquired information. The necessity calculator 62 may calculate the number of times of use of the lavatory equipment 40, for example, from the number of times of opening and closing detected by the opening and closing sensor of the door 25 of the private room 24, the number of times of locking and unlocking detected by the sensor that detects locking and unlocking of a lock installed in the door 25, the number of times of a user being detected by the pyroelectric sensor, the human sensor, the temperature sensor, or the like, the number of times of seating detected by the seating sensor of the toilet 30, the number of times of discharge detected by the drain sensor of the lavatory equipment 40, the time when the liquid soap is discharged by the liquid soap supply device of the washbasin 26, the number of times of discharge of water from the water faucet of the washbasin 26, the time when water is discharged from the water faucet, the operation information of each piece of lavatory equipment, and the like.

In Step S14, the necessity calculator 62 calculates the work necessity of each piece of lavatory equipment 40 based on the acquired information. The work necessity in some embodiments includes a cleaning necessity indicating a degree of necessity of cleaning the lavatory equipment 40, a replenishment necessity indicating a degree of necessity of replenishing a consumable for the lavatory equipment 40, and an abnormality removal necessity indicating a degree of necessity of removing an abnormality of the lavatory equipment 40. The necessity calculator 62 calculates the cleaning necessity based on a comparison between the number of times of use of the lavatory equipment 40 and a cleaning reference value related to cleaning. For example, the necessity calculator 62 calculates a ratio of the number of times of use to the cleaning reference value as the cleaning necessity. The necessity calculator 62 calculates the replenishment necessity based on a comparison between the number of uses of the lavatory equipment 40 and a replenishment reference value related to replenishment of a consumable. For example, the necessity calculator 62 calculates a ratio of the number of times of use to the replenishment reference value as the replenishment necessity. For example, when the reference value is 100 and the number of times of use is 50, the necessity calculator 62 calculates the work necessity to be 50%. The cleaning reference value and the replenishment reference value may be the same value or different values. The necessity calculator 62 calculates the abnormality removal necessity based on the detection of the abnormality of the lavatory equipment 40. For example, when it is detected that dirty water has overflowed from the water receiver of the lavatory equipment 40, the necessity calculator 62 sets the abnormality removal necessity to the maximum value (100%, for example, when the maximum value is set to 100%). The cleaning necessity, the replenishment necessity, and the abnormality removal necessity are collectively referred to as the work necessity below.

In Step S16, the generator 63 generates list information based on the calculated work necessity. For example, the generator 63 may generate the list information in units of the lavatory equipment 40 or the lavatory 21, or may generate the list information in units of the content of work.

In Step S18, the transmitter 64 transmits the list information to the person-in-charge terminal 90.

In Step S20, the list information display 97 of the person-in-charge terminal 90 displays a list of the work necessity of each piece of lavatory equipment 40 based on the list information transmitted from the management apparatus 50. An example of a list display screen in some embodiments will be described later.

When the person in charge of work completes the work of the lavatory equipment 40 and inputs a message that the work has been completed, to the person-in-charge terminal 90, in Step S22, the transmitter 98 of the person-in-charge terminal 90 transmits work record information including the message that the work has been completed, the content of the work, and the like to the management apparatus 50.

In Step S24, the work record information acquirer 65 acquires the work record information from the person-in-charge terminal 90, and stores the work record information in the work record information holder 83 as a work history of the lavatory equipment 40.

Thereafter, the process returns to Step S14, and the necessity calculator 62 newly calculates the work necessity of each lavatory equipment 40. In Step S16, the generator 63 generates the list information based on the newly calculated work necessity. In Step S18, the transmitter 64 transmits the latest list information to the person-in-charge terminal 90. In Step S20, the list information display 97 of the person-in-charge terminal 90 presents the latest list information output from the management apparatus 50 to the person in charge of work. The person in charge of work moves to the installation location of the lavatory equipment 40 to be worked next, and works on the lavatory equipment 40.

When a plurality of pieces of lavatory equipment 40 installed in the facility 20 are worked by a plurality of persons in charge of work, the pieces of lavatory equipment 40 assigned to the respective persons in charge of work may be determined in advance so as not to overlap with each other.

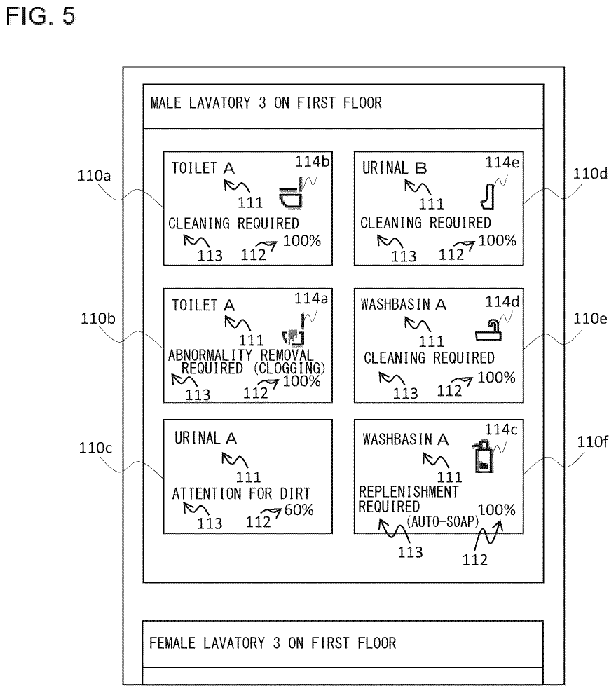

FIG. 5 illustrates an example of a display screen of the list information displayed by the person-in-charge terminal 90 according to some embodiments. In the example of FIG. 5, a list of sets of pieces of the lavatory equipment 40 in a male lavatory 3 on the first floor and the work necessity thereof are disposed in display regions 110a to 110f. Each of the display regions 110a to 110f includes an identification region 111 for identifying the lavatory equipment 40, a necessity display region 112 indicating the calculated work necessity, and a state display region 113 indicating the state of the lavatory equipment 40 based on the calculated work necessity. In the example of FIG. 5, the maximum value of each work necessity in the necessity display region 112 is indicated as 100%.

Each of the display regions 110a to 110f can display an indicator 114 indicating required work, when the work necessity is equal to or greater than a threshold value. For example, the threshold value is set to 90%. For example, in each of the display regions 110 excluding the display region 110c, any one of the cleaning necessity, the replenishment necessity, and the abnormality removal necessity is 100% and equal to or greater than the threshold value, and thus, the work being necessary is displayed by the indicator 114 in a highlighted manner or in an emphasized manner. In the display region 110c, the work necessity is 60% and is equal to or smaller than the threshold value. Thus, the indicator 114 is not displayed. An indicator 114a indicates that an abnormality needs to be removed due to an occurrence of overflow in the toilet 30. An indicator 114b indicates that the toilet 30 needs to be cleaned. An indicator 114c indicates that a liquid soap needs to be replenished. An indicator 114d indicates that the washbasin 26 needs to be cleaned. An indicator 114e indicates that the urinal 32 needs to be cleaned.

The display region 110a indicates that the cleaning necessity of the toilet A is 100% and the toilet A needs to be cleaned. The display region 110b indicates that the abnormality removal necessity of the toilet A is 100% and the abnormality of the toilet A needs to be removed. The display region 110c indicates that the cleaning necessity of the urinal A is 10%, and the urinal A is clean. The display region 110d indicates that the cleaning necessity of the urinal B is 100% and the urinal B needs to be cleaned. The display region 110e indicates that the cleaning necessity of the washbasin A is 100% and the washbasin A needs to be cleaned. The display region 110f indicates that the replenishment necessity of the liquid soap of the automatic soap in the washbasin A is 100% and the liquid soap of the automatic soap in the washbasin A needs to be replenished. Therefore, the person in charge of work who has referred to the display screen of the list information of FIG. 5 can recognize that the necessity of cleaning and abnormality removal for the lavatory A is high, the necessity of work for the urinal A is low, the necessity of cleaning for the urinal B is high, and the necessity of cleaning and replenishment for the washbasin A is high.

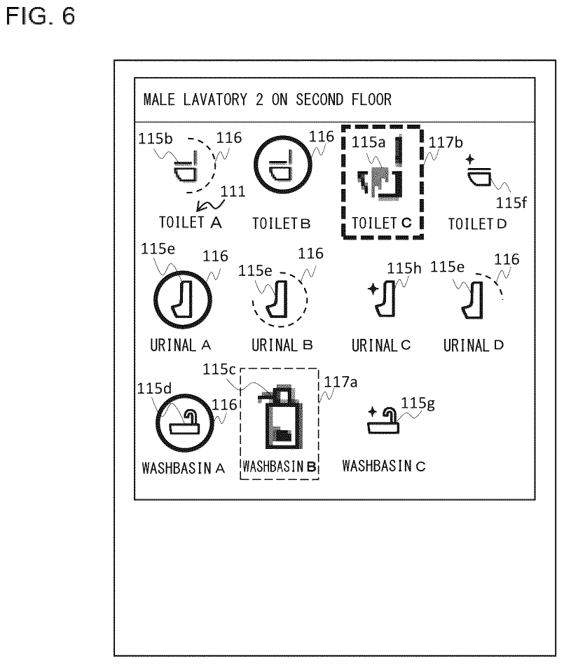

FIG. 6 illustrates another example of the display screen of the list information displayed by the person-in-charge terminal 90 according to some embodiments. In the example of FIG. 6, a list of sets of each piece of lavatory equipment 40 in a male lavatory 2 on the second floor and the work necessity thereof is displayed by using an indicator 115 indicating the type of the lavatory equipment 40 and the highlighted display of the lavatory equipment 40. An indicator 115a indicates that an abnormality needs to be removed due to an occurrence of overflow in the toilet 30. An indicator 115b indicates that it relates to the cleaning necessity of the toilet 30. An indicator 115c indicates that replenishment work of a liquid soap is necessary. An indicator 115d indicates that it relates to the cleaning necessity of the washbasin 26. An indicator 115e indicates that it relates to the cleaning necessity of the urinal 32. An indicator 115f indicates that the toilet 30 is in a clean state. An indicator 115g indicates that the washbasin 26 is in a clean state. An indicator 115h indicates that the urinal 32 is in a clean state. Here, the clean state is, for example, an unused state after cleaning is completed.

In the example of FIG. 6, the cleaning necessity of each piece of lavatory equipment 40 is displayed by a circular bar 116. For example, when the cleaning necessity is 50%, as in the example of the toilet A in FIG. 6, the circular bar 116 is displayed as a semicircle to indicate that the cleaning necessity is 50%. When the cleaning necessity is 100%, as in the example of the toilet B in FIG. 6, the circular bar 116 is displayed as a complete circle to indicate that the cleaning necessity is 100%. As the cleaning necessity increases, the degree of extension of the circular bar 116 is adjusted so that the circular bar 116 extends.

When the work necessity is equal to or greater than the threshold value, an image of the lavatory equipment 40 is displayed in a highlighted manner. For example, basically, the circular bar 116 is displayed by a dotted line (for example, the toilet A of FIG. 6). When the cleaning necessity is equal to or greater than the threshold value, an image of the toilet 30 is displayed in a highlighted manner by displaying the circular bar 116 with a thick solid line (for example, the toilet B of FIG. 6).

In the example of FIG. 6, replenishment of a consumable being necessary is displayed in a more highlighted manner than the case where cleaning is necessary. For example, when the replenishment necessity is equal to or greater than the threshold value, the characters in the identification region 111 are indicated in bold, the indicator 115c is indicated to be larger than usual, and the identification region 111 and the indicator 115c are surrounded by a first emphasis display frame 117a of a dotted line, whereby the washbasin 26 is displayed in a highlighted manner (for example, the washbasin B of FIG. 6). For example, for the washbasin B, the indicator 115b regarding the cleaning necessity of the washbasin 26 is not displayed, and only the indicator 115c indicating that replenishment is necessary is displayed.

The point that an abnormality needs to be removed is displayed in a more highlighted manner than the case where a consumable needs to be replenished. For example, when the abnormality removal necessity is equal to or greater than the threshold value, the characters in the identification region 111 are displayed in bold, the indicator 115a is displayed larger than usual, and the identification region 111 and the indicator 115a are surrounded by a second emphasis display frame 117b of a bold dotted line (for example, the toilet C of FIG. 6). Removal of an abnormality being necessary is displayed in a more highlighted manner than the case where cleaning is necessary. For example, for the toilet C, the indicator 115b regarding the cleaning necessity of the toilet 30 is not displayed, and only the indicator 115a indicating that an abnormality needs to be removed is displayed.

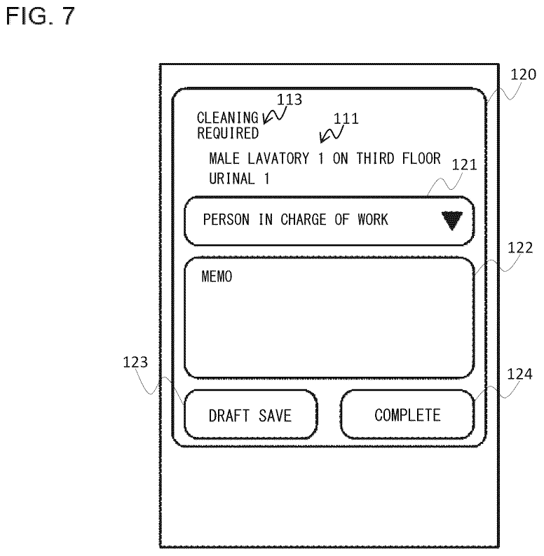

FIG. 7 illustrates an example of a display screen at the time of work completion, which is displayed by the person-in-charge terminal 90, according to some embodiments. When the worker completes the work on the lavatory equipment 40, for example, by pressing the display region 110 of FIG. 5 or the indicator 115 of FIG. 6 for the lavatory equipment 40 on which the work has been completed, the display screen shifts to a display screen 120 at the time of completion of the work for the lavatory equipment 40. The display screen 120 at the time of work completion in FIG. 7 includes an identification region 111, a state display region 113, a person-in-charge-of-work input region 121, a memo region 122, a draft save button 123, and a completion button 124. In the memo region 122, for example, the content of work, situations of the lavatory equipment 40 and the lavatory 21, presence or absence and the content of a trouble, information regarding a person in charge of work, and the like are input. After necessary items are input on the display screen at the time of work completion, the completion button 124 is pressed to complete the input indicating that the work has been completed, and each input item is transmitted to the management apparatus 50 as work record information.

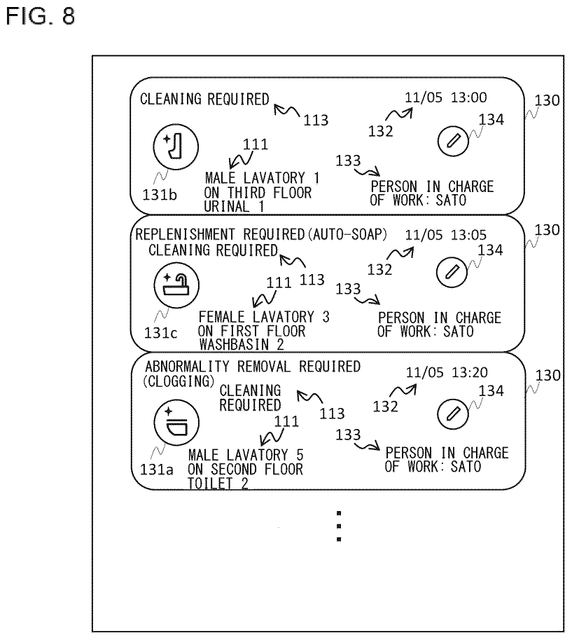

The work history and the information input by the person in charge of work may be able to be confirmed and corrected by the manager of the facility 20, the manager of work, another person in charge of work, or the like. FIG. 8 illustrates an example of a display screen of the work history displayed by the person-in-charge terminal 90. In the example of FIG. 8, the work history of the lavatory equipment 40 on which work has been completed is shown in each work history display region 130. Each work history display region 130 includes an identification region 111, a state display region 113, an indicator 131 indicating that work has been completed, a work completion date and time display region 132, a person-in-charge-of-work display region 133, and a memo viewing button 134. The work history display regions 130 are arranged side by side from the top in order of completion of the work. Indicators 131a to 131c in FIG. 8 indicate that the work on the toilet 30 has been completed, that the work on the urinal 32 has been completed, and that the work on the washbasin 26 has been completed, respectively. By pressing the memo viewing button 134, it is possible to view the contents input in the memo region 122 of FIG. 7 at the time of work completion.

In some embodiments, the management apparatus 50 generates list information for displaying a list of sets of the lavatory equipment 40 and the work necessity of the lavatory equipment 40, and transmits the list information to the person-in-charge terminal 90. According to some embodiments, the person in charge of work can grasp the work necessity of each piece of lavatory equipment 40 from the list, and thus it is possible to work in an appropriate order. As a result, it is possible to further improve the work efficiency for the lavatory equipment 40.

In some embodiments, the generator 63 generates the list information to display, in a highlighted manner, the lavatory equipment 40 of which at least one of the cleaning necessity, the replenishment necessity, and the abnormality removal necessity is equal to or greater than the threshold value. According to some embodiments, the person in charge of work can easily grasp the lavatory equipment 40 that needs work.

In some embodiments, the generator 63 generates the list information to display that cleaning is necessary in a highlighted manner when the cleaning necessity is equal to or greater than the threshold value, to display that a consumable needs to be replenished in a highlighted manner when the replenishment necessity is equal to or greater than the threshold value, and to display that an abnormality needs to be removed in a highlighted manner when the abnormality removal necessity is equal to or greater than the threshold value. According to some embodiments, the person in charge of work can easily grasp which work of cleaning, replenishment, and removal of abnormality is necessary.

In some embodiments, the generator 63 generates the list information to display that the consumable needs to be replenished and that the abnormality needs to be removed, in a more highlighted manner than that the cleaning is necessary. According to some embodiments, the person in charge of work can appropriately work according to the urgency of work.

In some embodiments, the generator 63 generates the list information to display that the abnormality needs to be removed, in a more highlighted manner than that the consumable needs to be replenished. According to some embodiments, the person in charge of work can appropriately work according to the urgency of work.

Modification examples will be described below.

In some embodiments, the work includes cleaning, replenishment of a consumable, and removal of an abnormality, but it is sufficient that the work includes cleaning and at least one of replenishment of a consumable and removal of an abnormality. In this case, the work necessity may include the cleaning necessity and at least one of the replenishment necessity and the abnormality removal necessity.

In some embodiments, when the replenishment necessity, the abnormality removal necessity, and the cleaning necessity are equal to or greater than the threshold value, the replenishment, the abnormality removal, and the cleaning being necessary is displayed in a highlighted manner. However, some embodiments are not limited to this. When at least one of the replenishment necessity, the abnormality removal necessity, and the cleaning necessity is equal to or greater than the threshold value, the highlighted display may be performed on that the work necessity is equal to or greater than the threshold value and the work is necessary.

In some embodiments, an example of managing the lavatory equipment 40 installed in the lavatory 21 of the facility 20 has been described. The technique in the present disclosure is also applicable to a case of managing a lavatory of a general house, a public lavatory installed in a park, a road, or the like.

In some embodiments, the lavatory equipment 40 includes the washbasin 26, the toilet 30, and the urinal 32. However, some embodiments are not limited to this. For example, a waste flushing unit or the like having a bowl portion for an ostomate to treat waste such as excrement. The lavatory equipment 40 may be any equipment that is installed in the lavatory 21, includes a water receiver that receives water discharged from a water discharger, and may overflow the water when the water receiver is clogged.

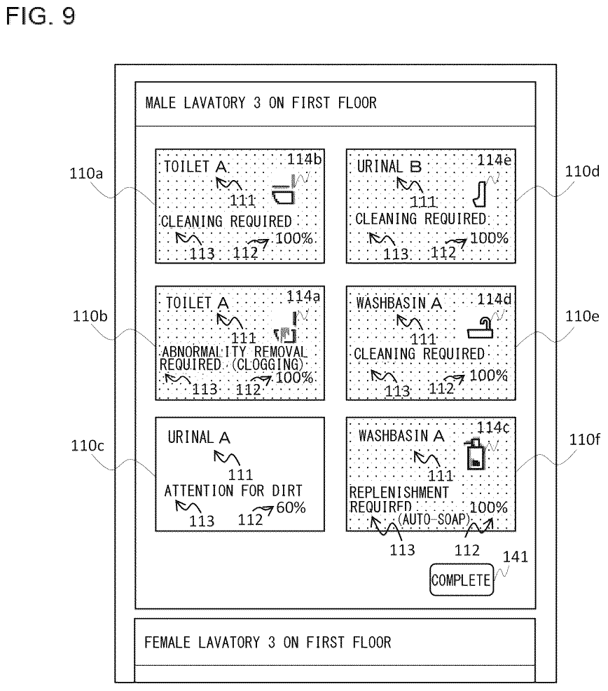

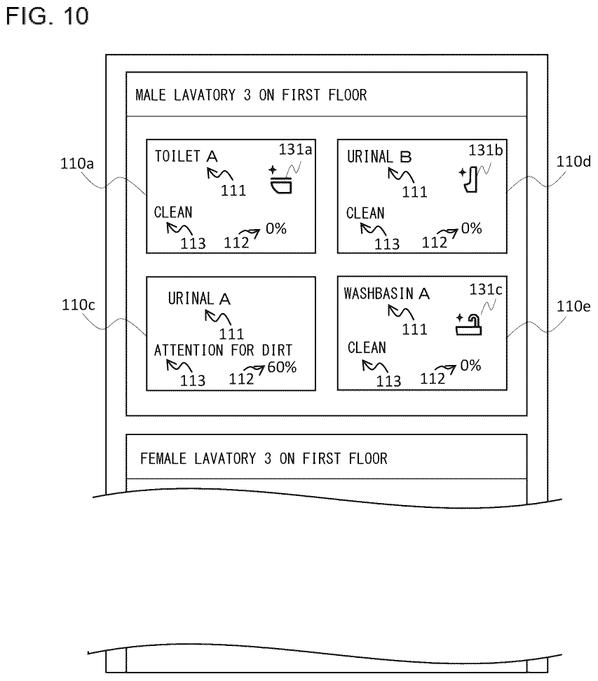

FIG. 9 illustrates another example of the display screen at the time of work completion, which is displayed by the person-in-charge terminal 90, according to some embodiments. The generator 63 may generate the list information to enable the person-in-charge terminal 90 to collectively transmit, to the management apparatus 50, the completion of work for the lavatory equipment 40 for which the work has been completed among pieces of the lavatory equipment 40 displayed in the list. According to some embodiments, it is possible to reduce time and effort to input that the work has been completed, by operating the screen of the person-in-charge terminal 90. For example, when the worker completes the work on the lavatory equipment 40, by pushing the inner portion of the display region 110 related to the lavatory equipment 40 for which the work has been completed, the inner portion of the display region 110 is represented in an unusual mode such as a dot pattern, and the corresponding lavatory equipment 40 is in a selected state. In the example of FIG. 9, the display regions 110a and 110b related to the toilet A, the display region 110d related to the urinal B, and the display regions 110e and 110f related to the washbasin A are selected. After completion of the selection, the completion button 141 is pressed, whereby a message that the work on each piece of the selected lavatory equipment 40 has been completed is transmitted to the management apparatus 50 as the work record information. Thereafter, the display screen is updated by newly generating the list information. FIG. 10 illustrates an example of a display screen reflecting work completion. In the example of FIG. 10, the indicator in the display region 110a for the toilet A is changed to the indicator 131a indicating that the work on the toilet 30 has been completed. The indicator in the display region 110d for the urinal B is changed to the indicator 131b indicating that the work on the urinal 32 has been completed. The indicator in the display region 110e for the washbasin A is changed to the indicator 131c indicating that the work on the washbasin 26 has been completed. The display region 110b related to the abnormality of the toilet A and the display region 110f related to the replenishment of the automatic soap of the washbasin A are deleted. As a result, the person in charge of work can easily grasp the lavatory equipment 40 on which work has been completed.

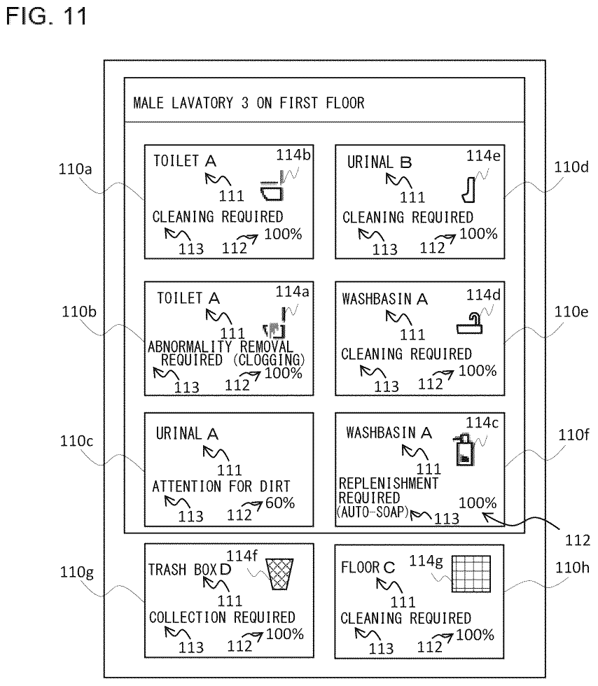

FIG. 11 illustrates still another example of the display screen of the list information, which is displayed by the person-in-charge terminal 90, according to some embodiments. The necessity calculator 62 may calculate a work necessity for an installation disposed outside the lavatory 21 in the facility 20. The generator 63 may generate the list information to further display a list of the installations having the work necessity that is equal to or greater than the threshold value. The work on the installation here is, for example, cleaning of a trash box and a floor, replenishment of a disinfectant, and the like. In the example of FIG. 11, the work necessity for a trash box D and an indicator 114f indicating that it is necessity to collect the garbage are displayed in a display region 110g. The work necessity for a floor C and an indicator 114g indicating that the floor needs to be cleaned are displayed in a display region 110h. For example, the necessity calculator 62 obtains the amount of trash in the trash box based on the detection result of a load sensor that detects the weight of the trash box, and calculates the work necessity so that the larger the amount, the higher the work necessity. For example, the necessity calculator 62 obtains the number of people who have passed through the floor from the detection result of the human sensor, and calculates the work necessity so that the larger the number of passing people, the higher the work necessity. For example, the necessity calculator 62 obtains the amount of a disinfectant of the disinfectant supply device based on the detection result of the load sensor that detects the weight of the disinfectant supply device, and calculates the work necessity so that the smaller the amount, the higher the work necessity. According to some embodiments, the person in charge of work can grasp the work necessity of each piece of lavatory equipment 40 and the installation disposed outside the lavatory 21 from the list, and thus it is possible to work in a more appropriate order. As a result, it is possible to further improve the work efficiency for the lavatory equipment 40 and the installation.

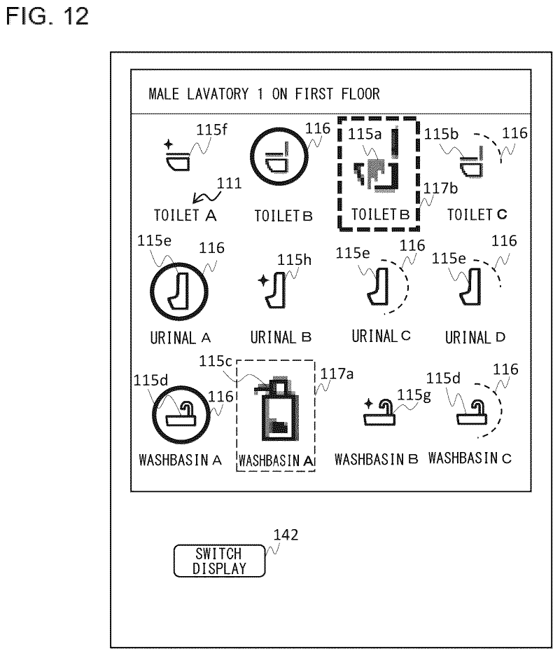

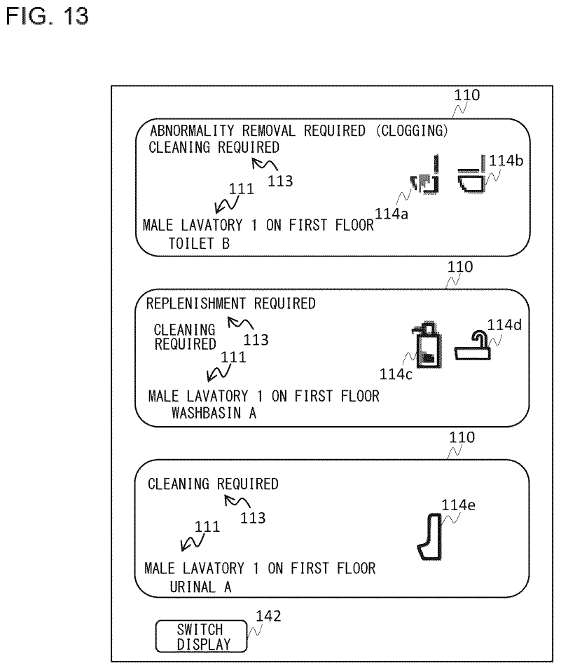

FIG. 12 illustrates still another example of the display screen of the list information, which is displayed by the person-in-charge terminal 90. In the example of FIG. 12, a first display screen in which a list of sets of each piece of lavatory equipment 40 in a male lavatory 1 on the first floor and the work necessity for the lavatory equipment 40 is displayed is illustrated. As the number of pieces of lavatory equipment 40 to be displayed increases, it becomes more difficult to grasp lavatory equipment 40 that needs work and the work. Therefore, the generator may generate list information including the content of the first display screen and the content of the second display screen on which a list of pieces of lavatory equipment 40 having the work necessity that is equal to or greater than the threshold value among the pieces of lavatory equipment 40 in the sets displayed in the list is displayed. The first display screen and the second display screen are switchable and displayable on the person-in-charge terminal 90 side. The first display screen of FIG. 12 includes a display switching button 142. By pressing the display switching button 142 in FIG. 12, the display content is switched to the second display screen on which the list of pieces of the lavatory equipment 40 having the work necessity that is equal to or greater than the threshold value among the pieces of lavatory equipment displayed in the list. FIG. 13 illustrates an example of the second display screen. In the example of FIG. 13, among the pieces of lavatory equipment 40 in the male lavatory 1 on the first floor, the lavatory equipment 40 having the work necessity that is equal to or greater than the threshold value is displayed in a list. The display screen can be switched to the first display screen in FIG. 12 by pressing the display switching button 142 in FIG. 13. According to some embodiments, it is possible to display only the lavatory equipment 40 having the work necessity that is equal to or greater than the threshold value as necessary, so that it is easy to grasp lavatory equipment 40 that needs work and the work. Therefore, it is possible to further improve convenience. The list information itself may include the first display screen and the second display screen, and a command for causing the person-in-charge terminal 90 to execute a process for switching screens between the first display screen and the second display screen. Alternatively, an application for creating the first display screen and the second display screen based on the work necessity may be installed on the person-in-charge terminal 90 in advance, and the list information may cause the person-in-charge terminal 90 to create the first display screen and the second display screen based on the work priority in the list information.

In some embodiments, the necessity calculator 62 calculates the ratio of the number of times of use with respect to the reference value as the work necessity. Some embodiments are not limited thereto, and a difference of the number of times of use from the reference value or the like may be calculated as the work necessity. In this case, the remaining number of times of use until the number of times of use of the lavatory equipment 40 reaches the reference value may be displayed by using this difference. The necessity calculator 62 may calculate the work necessity according to the number of times of use or the like without comparing the number of times of use to the reference value, that is, without using the reference value.

For example, the necessity calculator 62 may calculate the cleaning necessity based on the odors of the lavatory equipment 40, the degree of dirt, and the like. For example, the necessity calculator 62 may calculate the replenishment necessity according to the amount of toilet paper, the amount of liquid soap in the liquid soap supply device (not illustrated), and the amount of disinfectant in the disinfectant supply device (not illustrated). These amounts may be determined by a known method such as using a weight sensor.

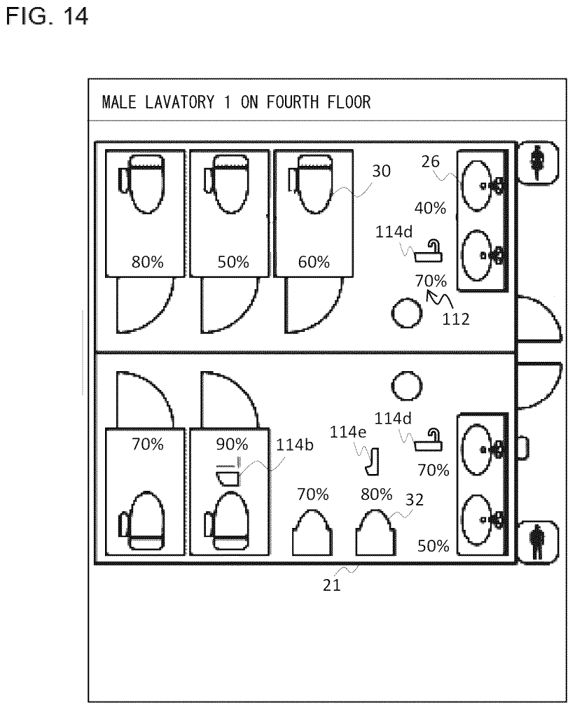

FIG. 14 illustrates still another example of the display screen of the list information, which is displayed by the person-in-charge terminal 90, according to some embodiments. In the example of FIG. 14, a set of each piece of lavatory equipment 40 in the male lavatory 1 on the fourth floor and the work necessity for the lavatory equipment 40 is displayed in a list in a map format. The generator 63 may generate the list information to display a list of sets of pieces of the lavatory equipment 40 and the work necessity by using the actual arrangement of the pieces of lavatory equipment 40 in the lavatory 21. As illustrated in FIG. 14, in the map of the lavatory 21, the work necessity is indicated in the necessity display region 112 of each piece of lavatory equipment 40, and the indicators 114b, 114d, and 114e are shown for the lavatory equipment 40 having the work necessity that is greater than the threshold value. According to some embodiments, since the correspondence relationship between the pieces of lavatory equipment 40 displayed in a list and the actual lavatory equipment 40 in the lavatory 21 can be easily understood, it is possible to further improve the work efficiency for the lavatory equipment 40.

In some embodiments, the threshold value is set to the same value in the washbasin 26, the toilet 30, and the urinal 32. However, some embodiments are not limited thereto, and the threshold value may be set to different values. For example, in FIG. 14, the threshold value for the washbasin 26 is set to 65%, the threshold value for the toilet 30 is set to 85%, and the threshold value for the urinal 32 is set to 75%. Each piece of lavatory equipment 40 has a different use mode and a different degree of dirt per use. Therefore, according to some embodiments, it is possible to more appropriately determine whether or not work is necessary.

The threshold value may be set to be different depending on the type of the lavatory 21 (for example, a male lavatory, a female lavatory, a multi-purpose lavatory, and a child lavatory). The use mode and the degree of dirt per use are also different for each lavatory 21. Therefore, according to some embodiments, it is possible to more appropriately determine whether or not work is necessary. The threshold value may be set differently depending on, for example, the model of the lavatory equipment 40 or the like, or may be automatically changed depending on the increase, decrease or congestion status of people in the facility 20, a floor 36, an area 35, and the lavatory 21, the time period, the weather around the facility 20, the number of claims received for the lavatory 21, the usage amounts or remaining amounts of toilet paper, liquid soap, disinfectant, the odors of the lavatory 21, and the like.

Although the work necessity of the lavatory equipment 40 is indicated for each lavatory 21, the present embodiment is not limited thereto. For example, the work necessity of the lavatory equipment 40 may be indicated for each facility 20, each floor 36, and each area 35.

In some embodiments, when an abnormality of the lavatory equipment 40 is detected, the abnormality removal necessity is set to the maximum value. However, some embodiments are not limited thereto. For example, it is assumed that, when a foreign matter adheres to the piping of the lavatory equipment 40, the flow velocity and the flow rate of discharged water of the lavatory equipment 40 decrease. Thus, the abnormality removal necessity may be calculated according to the flow velocity and the flow rate of the discharged water of the lavatory equipment 40.

In the examples of FIGS. 6 and 12, the cleaning necessity is displayed by the circular bar 116, but the replenishment necessity and the abnormality removal necessity may also be displayed by the circular bar 116. The work necessity may be displayed in various modes such as a linear bar instead of the circular bar 116.

The mode of the highlighted display of the lavatory equipment 40 is not limited to the examples of FIGS. 5 and 6 and the like. For example, the lavatory equipment 40 may be displayed in a highlighted manner by a difference in color, a color depth, discoloration, flashing, decorations (for example, star marks and the like), and the like.

The configuration of collectively transmitting the message that the work has been completed is not limited to the example of FIG. 9. The message that the work has been completed may be automatically collectively transmitted to the management apparatus 50 when a predetermined date and time condition such as a day of the week, a time, or a holiday is satisfied. For example, when the work system of a worker is set to complete the work on all pieces of the lavatory equipment 40 until a specific time such as 30 minutes before opening of a store, a message that the work has been completed for all pieces of the lavatory equipment 40 before 30 minutes before the opening of the store by using the date and time condition that is 30 minutes before the opening of the store is collectively transmitted to the management apparatus 50. As a result, it is possible to reduce time and effort to input that the work has been completed, by operating the screen of the person-in-charge terminal 90. The message that the work has been completed may be collectively transmitted to the management apparatus 50, for example, when predetermined work (for example, replenishment of toilet paper, or a special operation of lavatory equipment 40) has been performed, when it is detected that dirt is equal to or smaller than a predetermined criterion, when the person-in-charge terminal 90 is at a predetermined position, when image data of the lavatory equipment 40 on which the work has been completed is transmitted to the management apparatus 50 by the person-in-charge terminal 90, when a specific action pattern of the worker is detected after the special operation of the lavatory equipment 40, or the like. For example, pieces of the lavatory equipment 40 may be collectively selectable in units of the lavatory 21, the area 35, and the floor 36, or the message that the work on the selected lavatory equipment 40 has been completed may be collectively transmitted. According to some embodiments, it is also possible to reduce time and effort to input that the work has been completed, by operating the screen of the person-in-charge terminal 90.

A notification device including an LED lamp and the like may be provided in the vicinity of the washbasin 26, the toilet 30, and the urinal 32, and when the work necessity exceeds the threshold value, a notification of a message that work on the lavatory equipment 40 is required may be performed by blinking the LED lamp of the notification device. In this case, the notification device may be able to transmit the message that the work has been completed to the management apparatus 50 in a manner that the blinking of the LED lamp by the notification device turns off.

Although the present disclosure has been described above based on various embodiments, these embodiment merely illustrates the principle and application of the present disclosure. In addition, many modification examples and changes in arrangement of the embodiments can be made in a range without departing from the spirit of the present invention defined in the claims.

1. A management apparatus comprising:
an information acquirer that acquires information from at least one of a lavatory equipment or a sensor installed in a lavatory space;
a necessity calculator that calculates a work necessity indicating a degree of necessity of work on the lavatory equipment based on the acquired information;
a generator that generates list information for displaying a list of sets of the lavatory equipment and the work necessity for the lavatory equipment; and
a transmitter that transmits the list information to an external terminal device used by a person in charge of the work for the lavatory equipment.

2. The management apparatus of claim 1, wherein the work includes at least one of replenishment of a consumable and removal of an abnormality, and cleaning, and
wherein the work necessity includes at least one of a replenishment necessity indicating a degree of necessity of replenishing the consumable and an abnormality removal necessity indicating a degree of necessity of removing the abnormality, and a cleaning necessity indicating a degree of necessity of cleaning.

3. The management apparatus of claim 1, wherein
the generator generates the list information so that the terminal device displays, in a highlighted manner, an image of the lavatory equipment having the work necessity that is equal to or greater than a threshold value.

4. The management apparatus of claim 3, wherein the work includes replenishment of a consumable, removal of an abnormality, and cleaning,
wherein the work necessity includes a replenishment necessity indicating a degree of necessity of replenishing the consumable, an abnormality removal necessity indicating a degree of necessity of removing the abnormality, and a cleaning necessity indicating a degree of necessity of cleaning, and
wherein the generator generates the list information so as to highlight and display that the cleaning is necessary when the cleaning necessity is equal to or greater than the threshold value on the terminal device, so as to highlight and display that the consumable needs to be replenished when the replenishment necessity is equal to or greater than the threshold value on the terminal device, and so as to highlight and display that the abnormality needs to be removed when the abnormality removal necessity is equal to or greater than the threshold value on the terminal device.

5. The management apparatus of claim 4, wherein
the generator generates the list information so as to display, on the terminal device, that the consumable needs to be replenished and that the abnormality needs to be removed, in a more highlighted manner than that the cleaning is necessary.

6. The management apparatus of claim 5, wherein
the generator generates the list information so as to display, on the terminal device, that the abnormality needs to be removed, in a more highlighted manner than that the consumable needs to be replenished.

7. The management apparatus of claim 4, wherein the lavatory equipment includes a toilet, a urinal, and a washbasin, and
wherein the threshold value is enabled to be set to different values for the toilet, the urinal, and the washbasin.

8. The management apparatus of claim 7, wherein
the generator generates the list information so as to display the list of the sets on the terminal device by using an actual arrangement of the lavatory equipment in the lavatory space.

9. The management apparatus of claim 4, wherein
the threshold value varies depending on a type of the lavatory space.

10. The management apparatus of claim 1, wherein
the generator generates the list information so as to enable the terminal device to collectively transmit, to the management apparatus, completion of the work for the lavatory equipment for which the work has been completed among pieces of the lavatory equipment displayed in a list.

11. The management apparatus of claim 1, wherein the necessity calculator further calculates the work necessity of an installation disposed outside the lavatory space in a facility in which the lavatory space is provided, and
wherein the generator generates the list information so that the terminal device further displays a list of installations having the work necessity that is equal to or greater than a threshold value.

12. The management apparatus of claim 1, wherein
the generator generates the list information including a content of a first display screen on which the set is displayed in a list and a content of a second display screen on which pieces of the lavatory equipment having the work necessity that is equal to or greater than a threshold value among pieces of the lavatory equipment of the set displayed in a list are displayed in a list, the first display screen and the second display screen being switchable and displayable on the terminal device.

13. A non-transitory computer-readable storage medium storing a management program causing a computer to function as:
an information acquirer that acquires information from at least one of lavatory equipment and a sensor installed in a lavatory space;
a necessity calculator that calculates a work necessity indicating a degree of necessity of work on the lavatory equipment based on the acquired information;
a generator that generates list information for displaying a list of sets of the lavatory equipment and the work necessity for the lavatory equipment; and
a transmitter that transmits the list information to an external terminal device used by a person in charge of the work for the lavatory equipment.

14. A management system comprising:
a terminal device that is used by a person in charge of work on lavatory equipment installed in a lavatory space; and
a management apparatus that manages the work on the lavatory equipment, wherein
the management apparatus includes
an information acquirer that acquires information from at least one of the lavatory equipment and a sensor installed in a lavatory space,
a necessity calculator that calculates a work necessity indicating a degree of necessity of work on the lavatory equipment based on the acquired information,
a generator that generates list information for displaying a list of sets of the lavatory equipment and the work necessity for the lavatory equipment; and
a transmitter that transmits the list information to the terminal device, and
the terminal device includes a presenter that presents, to the person in charge of the work, the list information transmitted from the management apparatus.

15. A terminal device comprising:
a presenter that presents, to a person in charge of work, list information transmitted from a management apparatus that generates the list information for displaying a list of sets of lavatory equipment installed in a lavatory space and a work necessity indicating a degree of necessity of the work on the lavatory equipment based on information acquired from at least one of the lavatory equipment and a sensor.

16. A non-transitory computer-readable storage medium storing a management program causing a computer to function as:
a presenter that presents, to a person in charge of work, list information transmitted from a management apparatus that generates the list information for displaying a list of sets of lavatory equipment installed in a lavatory space and a work necessity indicating a degree of necessity of the work on the lavatory equipment based on information acquired from at least one of the lavatory equipment and a sensor.
